SEQUENCE LISTING (11 sequences)

SEQ ID NO: 1 - DNA, 17 bp, Artificial Sequence
  3′ acceptor probe
agatcggaagagcacac

SEQ ID NO: 2 - DNA, 17 bp, Artificial Sequence
  5′ phosphorylated donor probe
ggagctgtcgttcactc

SEQ ID NO: 3 - DNA, 30 bp, Artificial Sequence
  A first detection probe
caagtatgcagcgcgattgaccgtctcgtt

SEQ ID NO: 4 - DNA, 30 bp, Artificial Sequence
  A second detection probe
cgcaacgcttgggacggttccaatcggatc

SEQ ID NO: 5 - DNA, 30 bp, Artificial Sequence
  A third detection probe
acaaatccgaccagatcggacgatcatggg

SEQ ID NO: 6 - DNA, 30 bp, Artificial Sequence; identical to SEQ ID NO: 3
  A fourth detection probe

SEQ ID NO: 7 - DNA, 20 bp, Artificial Sequence
  3′ acceptor probe that specifically hybridizes GAPDH target
  misc_feature (19)..(20): 3′ terminal ribonucleotide
ttgagcacagggtactttat

SEQ ID NO: 8 - DNA, 20 bp, Artificial Sequence
  5′ phosphorylated donor probe that specifically hybridizes GAPDH target
actggtctcaagtcagtgta

SEQ ID NO: 9 - DNA, 20 bp, Artificial Sequence
  3′ acceptor probe that specifically hybridizes beta-actin target
  misc_feature (19)..(20): 3′ terminal ribonucleotide
aaggtgtgcacttttattca

SEQ ID NO: 10 - DNA, 20 bp, Artificial Sequence
  5′ phosphorylated probe that specifically hybridizes beta-actin target
actggtctcaagtcagtgta

SEQ ID NO: 11 - DNA, 32 bp, Artificial Sequence
  bridge oligo primer
  misc_feature (19)..(20): nucleotides that contain a phophorothioate bond substitution, which renders them less susceptible to degradation by nucleases
agtgaacgacagctccgtgtgctcttccgatc

(19) United States
(12) Patent Application Publication
(10) Pub. No.: US 2023/0039899 A1
(43) Pub. Date: Feb. 9, 2023

(54) IN SITU RNA ANALYSIS USING PROBE PAIR LIGATION

(71) Applicant: The Johns Hopkins University, Baltimore, MD (US)

(72) Inventors: Harry Larman, Baltimore, MD (US); Joel Credie, Gaithersburg, MD (US)

(21) Appl. No.: 17/790,293

(22) PCT Filed: Jan. 4, 2021
(86) PCT No.: PCT/US2021/012099

§ 371 Date: Jun. 30, 2022

Related U.S. Application Data
(60) Provisional application No. 62/956,691, filed on Jan. 3, 2020.

(51) Int. Cl. C12Q 1/6841; C12Q 1/6844

(52) U.S. Cl. CPC C12Q 1/6841; C12Q 1/6844

(57) ABSTRACT

The present invention relates to the field of ribonucleotide analysis. More specifically, the present invention provides compositions and methods for detection for nucleic acids using probe pair litigation. In particular embodiments, the compositions and methods of the present invention utilize a probe set comprising (1) a first multi-partite probe comprising a 5′ phosphorylated donor probe and a first bridge probe, wherein the 5′ phosphorylated donor probe specifically hybridizes to a target nucleic acid; and (ii) a second multi-partite probe comprising a 3′ acceptor probe and a second bridge probe, wherein the 3′ acceptor probe specifically hybridizes to the target nucleic acid adjacent to the 5′ donor probe and the second bridge probe is 5′ phosphorylated.

### CROSS-REFERENCE TO RELATED APPLICATIONS

This application claims the benefit of U.S. Provisional Application No. 62/956,691, filed Jan. 3, 2020, which is incorporated herein by reference in its entirety.

### GOVERNMENT SUPPORT CLAUSE

This invention was made with government support under grant no. CA202875 awarded by the National Institutes of Health. The government has certain rights in the invention.

### FIELD OF THE INVENTION

The present invention relates to the field of ribonucleotide analysis. More specifically, the present invention provides compositions and methods for in situ RNA analysis using probe pair litigation.

### INCORPORATION-BY-REFERENCE OF MATERIAL SUBMITTED ELECTRONICALLY

This application contains a sequence listing. It has been submitted electronically via EFS-Web as an ASCII text file entitled “P16123-02_ST25.txt.” The sequence listing is 2,942 bytes in size, and was created on Dec. 28, 2020. It is hereby incorporated by reference in its entirety.

### BACKGROUND OF THE INVENTION

Traditional gene expression methods that use bulk RNA analysis lack the ability to resolve transcript location, and thus fail to capture the inherent cellular heterogeneity present in virtually every tissue. Abundance and localization of RNA transcripts mapped to specific regions of tissue can complement histological analysis, providing an additional layer of molecular information¹. Subcellular resolution of mRNA abundance and location can in some cases be used to categorize biologically important cell-to-cell variability and interactions²⁻⁴.

The tumor microenvironment (TME) describes the cellular composition surrounding solid tumors with a specific focus on the immune cell composition in this particular region of interest. Antibody-based methods (immunofluorescence-IHC, immunohistochemistry-IF) measure specific protein expression profiles that help to determine the immune cell composition of the TME. While useful, IHC and IF analyses are often limited in specificity (due to antibody cross-reactivity) and are challenging to multiplex⁵. Technologies that alternatively quantify RNA in situ have therefore emerged as complementary to antibody-based assays. In situ detection of RNA within the TME can provide highly multiplexed measurements with spatial precision. Also, several diseases have disruptions of mRNA localization as a defining feature (e.g., spinal muscular atrophy, amyotrophic lateral sclerosis); it is thus necessary to develop improved methods that capture RNA abundance with spatial resolution^{6,7}.

Several methods and technology platforms have been developed to measure both RNA abundance and spatial location in situ. Fluorescence In Situ Hybridization (FISH) and derivatives of this technique, which use serial reprobing (e.g., seqFISH, MERFISH), while promising, have failed to move beyond certain technical limitations such as multiplexing limits due to molecular crowding (<hundred targets measured), the need for dedicated imaging platforms with low sample throughput and high costs (>40 probes required per transcript)⁸⁻¹¹. In situ sequencing (e.g., FISSEQ, Bar-Seq) of RNA molecules, like FISH-based methods, also suffer from many of the same technical challenges^{12,13}. scRNA-seq based methods (e.g., Drop-seq, Slide-seq) can profile whole transcriptomes, yet at high per sample cost (>$10,000) due to sequencing depth requirements combined with the computational difficulty in linking transcriptomes back to cellular locations prevents these methods from being widely adopted¹⁴⁻¹⁶. In these techniques, spatial resolution is determined by pixel size, which may not be of sufficient resolution (e.g., single cell) for certain applications. Recently shown to be a robust highly multiplexed method for detection of RNA, LISH (Ligation In situ Hybridization) is a probe ligation-based technology that has not yet been adopted for use in measuring both the abundance and transcript position in situ.¹⁸

### SUMMARY OF THE INVENTION

The present invention is based, at least in part, on the development of a multiplexed probe ligation method termed “LISH-Lock'n'Roll.” In particular embodiments, LISH-Lock'n'Roll can be used to fluorescently track RNA abundance and position with minimal cost, uses common laboratory instrumentation and requires a simple single-day workflow. The high level of signal amplification with LISH-Lock'n'Roll using a single probe set provides several unique advantages over single molecule FISH (smFISH)-based and in situ sequencing methods. First, in particular embodiments, robust amplification of the LISH-Lock'n'Roll probes, makes it possible to detect any RNA sequence in situ with a single probe set instead of the >40 probes/target required for methods such as MERFISH. Second, in certain embodiments, because LISH-Lock'n'Roll uses a single target-identifying probe, greater discrimination of RNA sequences of interest is possible based on the presence or absence of single polynucleotide polymorphisms (SNPs), mutations, novel splicing isoforms, and fusions. Third, LISH-Lock'n'Roll can be accomplished in a single day unlike other methods (e.g., FISSEQ), which may require several days to several weeks to complete, a trait, which can impact throughput. LISH-Lock'n'Roll's greatest commercial advantage is its simplicity and cost, features that will certainly foster its widespread adoption among academic and clinical labs beyond other in situ transcriptome platforms.

Accordingly, the present invention provides compositions and methods for detecting nucleic acids. In one aspect, the present invention provides compositions and methods for detecting ribonucleic acids. In another aspect, the present invention can be used to detect deoxyribonucleic acids. It is understood that embodiments reciting the detection of RNA is applicable to the detection of DNA.

In one embodiment, a method for detecting an immobilized target ribonucleic acid (RNA) comprises the steps of (a) contacting a biological sample comprising the target RNA in a reaction mixture with at least one probe set comprising (i) a first multi-partite probe comprising a 5′ phosphorylated donor probe and a first bridge probe, wherein the 5′ phosphorylated donor probe specifically hybridizes to the target RNA; and (ii) a second multi-partite probe comprising a 3′ acceptor probe and a second bridge probe, wherein the 3′ acceptor probe specifically hybridizes to the target RNA adjacent to the 5′ donor probe and the second bridge probe is 5′ phosphorylated; (b) incubating the reaction mixture of step (a) under conditions that permit hybridization of the at least one probe set to the target RNA present in the biological sample; (c) washing away unbound probe sets; (d) ligating the 5′ phosphorylated donor probe and the 3′ acceptor probe; (e) contacting the reaction mixture with at least one bridge primer that specifically hybridizes to the first bridge probe and the second bridge probe, wherein the first bridge probe and the second bridge probe anneal to the bridge primer adjacent to each other; (f) ligating the first bridge probe and the second bridge probe thereby forming a circularized probe that is hybridized to the target RNA; (g) amplifying the circularized probe by rolling circle amplification; and (h) detecting the target RNA.

It is understood that more than one probe set, each targeting a specific and distinct RNA, can be used in the present invention. In particular embodiments, the at least one probe set is configured for multiplex detection of 1-30,000 distinct target RNAs.

In certain embodiments, the multipartite probes range in size from 30-1000 nucleotides.

The target RNA can be a viral RNA, a bacterial RNA, a fungal RNA, a nematode RNA, a human RNA, a non-human mammal RNA, a non-mammalian animal RNA or combinations thereof.

In particular embodiments, the present invention is used to detect an immobilized target RNA. In a specific embodiment, the RNA is immobilized as part of a fixed biological sample comprising the target RNA. The fixed biological sample can comprise fixed tissue, frozen-fixed tissue, formalin fixed paraffin embedded tissue, adherent fixed cells, suspension fixed cells or fixed cells.

In alternative embodiments, the target RNA is immobilized by capture prior to washing step (c). A labeled target RNA capture probe can be used including, but not limited to, biotin, diogexin, acrydite, haloalkane, or click chemistry. A capture element can include avidin, streptavidin, neutravidin, anti-digoxin antibodies, click chemistry, halo protein, or a combination thereof. A solid support can be used to capture target RNA, and can include magnetic material, polystyrene, agarose, silica, lateral flow strip, microfluidic chambers, or a combination thereof can be used in the immobilization process.

In certain embodiments, the 3′ acceptor probe comprises at least one 3′ terminal ribonucleotide.

In other embodiments, contacting step (e) is performed prior to ligating step (d). In such embodiments, ligating steps (d) and (f) can be performed simultaneously. In another embodiment, contacting step (e) and ligating step (f) are performed prior to ligating step (d).

In particular embodiments, ligating step (d) is performed using a ligase selected from the group consisting of T4 RNA Ligase 2 (Rnl2), a Chlorella virus DNA ligase (PBCV-1 DNA Ligase), a T4 DNA Ligase, derivatives thereof, and combinations thereof.

In certain embodiments, detecting step (h) comprises sequencing of rolling circle amplification products. In a specific embodiment, the probe set comprises a barcode unique to the target RNA and wherein sequencing of the barcode detects the target RNA. In a more specific embodiment, the sequencing comprises sequencing by synthesis or sequencing by ligation. In an even more specific embodiment, the method is performed in situ on a fixed sample. In an alternative embodiment, the sequencing comprises sequencing by synthesis, and the synthesized sequence creates a unique color barcode that detects the target RNA.

In other embodiments, detecting step (h) comprises sequencing the ligated sequence formed by the donor probe and the acceptor probe.

In yet another embodiment, detecting step (h) comprises contacting the reaction mixture with a detectably labeled detector probe that specifically hybridizes the ligated sequence formed by the donor probe and the acceptor probe.

In other embodiments, the first multi-partite probe and the second multi-partitie probe each further comprise at least one detection probe. In a specific embodiment, the first multi-partite probe and the second multi-partite probe each comprise two detection probes. In an even more specific embodiment, the first multi-partite probe and the second multi-partite probe each comprise a spacer sequence between the two detection probes.

In such embodiments, detecting step (h) comprises contacting the reaction mixture with at least one detector probe that specifically hybridizes to the at least one detection probe and imaging the at least one detectable labeled detector probe. In particular embodiments, the at least one detector probe is detectably labeled. In a specific embodiment, the method can further comprise the step of identifying the location of the target RNA in the sample. In another embodiment, the method further comprises the step of quantifying the target RNA in the sample.

In particular embodiments, amplification step (g) is performed using a strand displacing DNA polymerase. In specific embodiments, the strand displacing DNA polymerase comprises Phi29 polymerase or Bst polymerase.

In another aspect, the present invention provides kits. In one embodiment, a kit comprises a probe set comprising (i) a first multi-partite probe comprising a 5′ phosphorylated donor probe and a first bridge probe, wherein the 5′ phosphorylated donor probe specifically hybridizes to a target RNA; and (ii) a second multi-partite probe comprising a 3′ acceptor probe and a second bridge probe, wherein the 3′ acceptor probe specifically hybridizes to the target RNA adjacent to the 5′ donor probe and the second bridge probe is 5′ phosphorylated. It is understood that the kit can comprises multiple probe sets targeting distinct RNAs.

In another embodiment, the kit further comprises a bridge primer that specifically hybridizes to the first bridge probe and the second bridge probe, wherein the first bridge probe and the second bridge probes anneal to the bridge primer adjacent to each other.

In other embodiments, the first multi-partite probe and the second multi-partitie probe each further comprise at least one detection probe. In a specific embodiment, the first multi-partite probe and the second multi-partite probe each comprise two detection probes. In a more specific embodiment, the first multi-partite probe and the second multi-partite probe each comprise a spacer sequence between the two detection probes.

In other embodiments, the kit further comprises at least one detector probe that specifically hybridizes to the at least one detection probe. In specific embodiments, the at least one detector probe is detectably labeled.

In particular embodiments, the multipartite probes range in size from 30-1000 nucleotides. In other embodiments, the 3′ acceptor probe comprises at least one 3′ terminal ribonucleotide.

The kit can further comprise a ligase for ligating the 5′phosphorylated donor probe and the 3′ acceptor probe. More specifically, the ligase comprises T4 RNA Ligase 2 (Rnl2), a Chlorella virus DNA ligase (PBCV-1 DNA Ligase), a T4 DNA Ligase, and derivatives thereof.

The kit can further comprise a ligase for ligating the first bridge probe and the second bridge probe to form a circularized probe that is hybridized to the target RNA.

In other embodiments, the kit further comprises a strand displacing DNA polymerase for amplifying by rolling circle amplification a circularized probe formed by ligating the first bridge probe and the second bridge probe and hybridized to the target RNA. The strand displacing DNA polymerase can comprise Phi29 polymerase or Bst polymerase.

### BRIEF DESCRIPTION OF THE FIGURES

FIG. 1A-1B. LISH-Lock'n'Roll probe set compositions. Probes are represented in their closed (locked) form. Blue indicates the 20+20 nt LnR sequences (target), orange represents individual 30 nt detector sequences (FIG. 1A—two detector sequences, FIG. 1B—four detector sequences, 2 per probe). Light green indicates 4 nt spacer sequences and green represents the combined 34 nt bridge sequence (17 nt contributed by each probe), finally red indicates the position of the diribonucleotide. Dashed line divides the locked probes into their respective 3′ acceptor and 5 donor probe halves.

FIG. 2. LISH-Lock'n'Roll workflow. In step 1, fixed cells or tissues are incubated probe sets, which hybridize to their targeted RNA sequences. Step 2 includes the washing away of unbound or partially annealed probes, followed by ligation of adjacent probe pairs via Rnl2. In step 3, samples are incubated with a universal bridge primer, which hybridizes all ligated probe pairs' bridge sequences. Step 4 includes ligation of the two 17 nt bridge sequences via T4 DNA ligase, resulting in a circularized probe set that is now locked in place. In step 5, samples are incubated with Phi29 for rolling circle amplification (RCA) of the locked circle, a reaction which is primed by the annealed bridge primer. In step 6, hybridization of fluorophore-labeled detector probes to the RCA products. Lastly, samples are imaged and deconvoluted to acquire the fluorescent code at each individual spot, allowing target quantity and location to be determined.

FIG. 3. Target multiplexing. Diagram of target multiplexing in which multiple probe sets, with each set with a distinct detector sequence allows simultaneous detection of two or more targets.

FIG. 4. Color barcoding. Diagram of a probe set having multiple distinct detector sequences to allow the creation of a fluorescent barcode to describe individual targets.

FIG. 5A-5C. Representative image and quantification of target multiplexing. FIG. 5A: Image of fixed cells with probe sets that target GAPDH (detector-1-Alexa-488, green) and β-actin (detector-2-Alexa-647, magenta) with nuclei highlighted by DAPI (blue). White box denotes zoomed-in region (below). Shown are the individual detector probes used (left, center) and as an overlay (right). Dashed lines indicate cell boundaries. FIG. 5B: Quantification by qPCR of recovered LISH-Lock'n'Roll products showing the increased signal achieved through rolling circle amplification (+Phi29-solid bars) by Phi29 polymerase over samples that were locked but not amplified by RCA (−Phi29-dashed bars). FIG. 5C: Characteristics of LISH-Lock'n'Roll spots: per cell averaged spot sizes and spot numbers for individual target probe sets.

FIG. 6. Signal amplification through Phi29-mediated rolling circle amplification. Image of fixed cells with probe sets that target GAPDH (detector-1-Alexa-488, green) and β-actin (detector-2-Alexa-647, magenta) with nuclei highlighted by DAPI (blue). With Phi29 RCA (left image) and without Phi29 RCA (right image).

FIG. 7A-7B. Representative image and quantification of color barcoding. FIG. 7A: Image of fixed cells with a probe set targeting β-actin that has four distinct detector sequences (two per probe). Two fluorescently labeled detector probes (one per probe) were used for hybridization of the RCA product (detector-1 probe-Alexa-488, green and detector-2 probe-Alexa-647, magenta). Nuclei are stained by DAPI (blue). White box denotes zoomed-in regions (below) represented as the individual detector probes used (left, center) and as an overlay (right). Dashed lines indicate cell boundaries. FIG. 7B: Quantification by qPCR of recovered LISH-Lock'n'Roll products showing the increased signal achieved through rolling circle amplification (+Phi29-solid bars) by Phi29 polymerase over samples that were locked but not amplified by RCA (−Phi29-dashed bars).

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. Definitions

“Detect” refers to identifying the presence, absence, or amount of the nucleic acid (e.g., RNA) to be detected.

By “detectable label” is meant a composition that when linked to a molecule of interest renders the latter detectable, via, for example, spectroscopic, photochemical, biochemical, immunochemical, or chemical means. For example, useful labels may include radioactive isotopes, magnetic beads, metallic beads, colloidal particles, fluorescent dyes, electron-dense reagents, enzymes (for example, as commonly used in an ELISA), biotin, digoxigenin, or haptens.

By “fragment” is meant a portion of a nucleic acid molecule or polypeptide. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “marker” is meant any protein or polynucleotide having an alteration in expression level or activity that is associated with a disease or disorder. The term “biomarker” is used interchangeably with the term “marker.”

By “multi-partite” is meant having several or many parts or divisions.

By “multi-partite probe set” is meant a probe set having multiple parts or divisions. As an example, a multi-partite probe set of the present invention may comprise (i) a first multi-partite probe comprising a 5′ phosphorylated donor probe, at least one detection probe and a first bridge probe, wherein the 5′ phosphorylated donor probe specifically hybridizes to the target RNA and (ii) a second multi-partite probe comprising a 3′ acceptor probe, at least one detection probe and a second bridge probe, wherein the 3′ acceptor probe specifically hybridizes to the target RNA adjacent to the 5′ donor probe and the second bridge probe is 5′ phosphorylated.

By “pathogen” is meant anything that can produce a disease including a bacterium, virus, fungi or other microorganism, as examples.

By “infection” is meant the invasion of an organism's body by disease-causing agents, their multiplication and the reaction of the host to these organisms and the toxins they produce. The infection may be caused by any microbes/microorganisms, including for example, bacteria, fungi, and viruses. Microorganisms can include all bacterial, Archaean, and the protozoan species. This group also contains some species of fungi, algae, and certain animals. In some embodiments, viruses may be also classified as microorganisms.

By “reduces” is meant a negative alteration of at least 10%, 25%, 50%, 75%, or 100%.

By “reference” is meant a standard or control conditions such as a sample (human cells) or a subject that is a free, or substantially free, of an agent such as a pathogen.

By “reference sequence” is meant a defined sequence used as a basis for sequence comparison. A reference sequence may be a subset of or the entirety of a specified sequence; for example, a segment of a full-length cDNA, RNA, or gene sequence, or the complete cDNA, RNA, or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 40 nucleotides, preferably at least about 60 nucleotides, more preferably at least about 75 nucleotides, and even more preferably about 100 nucleotides or about 300 nucleotides or any integer thereabout or there between.

By “sensitivity” is meant a percentage of subjects correctly identified as having a particular disease or condition, or pathogen.

By “specificity” is meant a percentage of subjects correctly identified as NOT having a particular disease or condition, or pathogen, i.e., normal or healthy subjects.

By “specifically binds” is meant a multi-partite probe set that recognizes and binds a nucleotide sequence of the invention, but which does not substantially recognize and bind other molecules in a sample, for example, a biological sample, which naturally includes nucleotide sequences unrelated to the invention. In some embodiments, a 5′ phosphorylated donor probe and a 3′ acceptor probe specifically hybridize or bind to a target RNA. In other embodiments, a genotyping probe specifically binds a target nucleic acid having a particular single nucleotide polymorphism (SNP), but does not specifically bind a nucleic acid having an alternative SNP.

By “subject” is meant any individual or patient to which the method described herein is applied. Generally, the subject is human, although as will be appreciated by those in the art, the subject may be an animal (e.g., pet, agricultural animal, wild animal, etc.), disease vector (e.g., mosquitoes, sandflies, triatomine bugs, blackflies, ticks, tsetse flies, mites, snails, lice, etc.), or an environmental sample (e.g., sewage, food products, etc.). Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

Nucleic acid molecules useful in the methods of the invention need not be 100% identical with an endogenous nucleic acid sequence, but will typically exhibit substantial identity. Polynucleotides having “substantial identity” to an endogenous sequence are typically capable of hybridizing with a target molecule. By “hybridize” is meant pair to form a double-stranded molecule between complementary polynucleotide sequences, or portions thereof, under various conditions of stringency. See, e.g., Wahl, G. M. and S. L. Berger (1987) Methods Enzymol. 152:399; Kimmel, A. R. (1987) Methods Enzymol. 152:507.

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and sometimes above 50° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Sequencing” or any grammatical equivalent as used herein may refer to a method used to sequence the amplified target nucleic acid proxy. The sequencing technique may include, for example, Next Generation Sequencing (NGS), Deep Sequencing, mass spectrometry-based sequence or length analysis, or DNA fragment sequence or length analysis by gel electrophoresis or capillary electrophoresis. Compatible sequencing techniques may be used including single-molecule real-time sequencing (Pacific Biosciences), Ion semiconductor (Ion Torrent sequencing), pyrosequencing (454), sequencing by synthesis (Illumina), sequencing by ligation (SOLiD sequencing), chain termination (Sanger sequencing), Nanopore DNA sequencing (Oxford Nanosciences Technologies), Helicos single molecule sequencing (Helicos Inc.), sequencing with mass spectrometry, DNA nanoball sequencing, sequencing by hybridization, and tunneling currents DNA sequencing.

By “NGS” is meant Next Generation Sequencing. NGS platforms perform massively parallel sequencing, during which millions of fragments of DNA from a single sample are sequenced in unison. Massively parallel sequencing technology facilitates high-throughput sequencing, which allows an entire genome to be sequenced in less than one day. The creation of NGS platforms has made sequencing accessible to more labs, rapidly increasing the amount of research and clinical diagnostics being performed with nucleic acid sequencing.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

“Primer set” means a set of oligonucleotides that may be used, for example, in a polymerase chain reaction (PCR). A primer set comprises at least 2, 4, 6, 8, 10, 12, 14, 16, 18, 20, 30, 40, 50, 60, 80, 100, 200, 250, 300, 400, 500, 600, or more primers.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, the term “sub-probe” may refer to any of the two or more probes that bind the contiguous target sequence without leaving any unbound intervening nucleotides. In some embodiments, the multi-partite probe described herein may include at least two “sub-probes.” In another embodiment, each of the at least two sub-probes of the plurality of multi-partite probes may be about 10-50 nucleotides in length. Once the probes are ligated, the ligated multi-partite probe (alternatively, the “ligated sub-probe”) may be released from the RNA. In some embodiments, the sub-probe may contain appended primer binding site (e.g., adapters) to facilitate subsequent amplification of the target nucleic acid proxy. In other embodiments, at least one of the two or more sub-probes may be referred to as “acceptor sub-probes” that have a 3′-termination of at least two RNA bases.

As used herein, “appended primer binding” sites may refer to binding sites within the multi-partite probe or sub-probes described herein that facilitate amplification of the target nucleic acid proxy. “Appended primer binding sites” may also be referred to as “adapters.”

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### II. LISH-Lock'n'Roll

Hybridization of probes in a sample, followed by in situ ligation (“LISH), locks specifically circularized probe set around an RNA target sequence. Rolling circle amplification (“LISH-Lock'n'Roll”), followed by fluorescently labeled detector probe hybridization, enables simultaneous in situ quantification and localization of RNA sequences with subcellular precision. This technology is a time and cost-effective alternative to other in situ RNA analysis methods.

In prior work, the inventors have described the utility of T4 RNA Ligase 2 (Rnl2), an enzyme that performs RNA-templated ligations of DNA probes with very high efficiency, when the two 3′ bases of the acceptor probe are composed of ribonucleotides^{17,18}. This ligation chemistry enables the multiplexed quantification of RNA in a high throughput assay referred to as RNA-mediated oligonucleotide Annealing, Selection, and Ligation with sequencing (‘RASL-seq’). The present inventors have also applied this ligation chemistry to the analysis of RNA sequences in formalin fixed tissue specimens in an assay called Ligation In Situ Hybridization sequencing (LISH-seq). Here, the inventors present LISH-Lock'n'Roll, which is a novel, yet related approach for multiplexed quantification and localization of RNA sequences in a fixed biological specimen. Target RNA sequences are preferably greater than 40 nucleotides long and may be associated with host or infectious disease-specific transcripts.

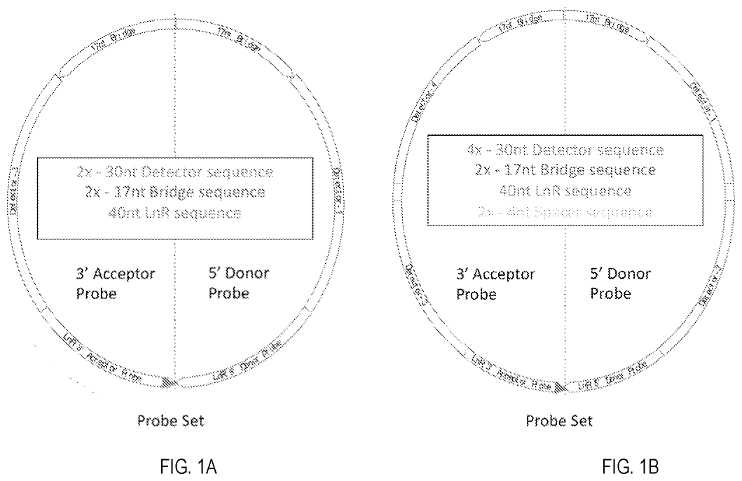

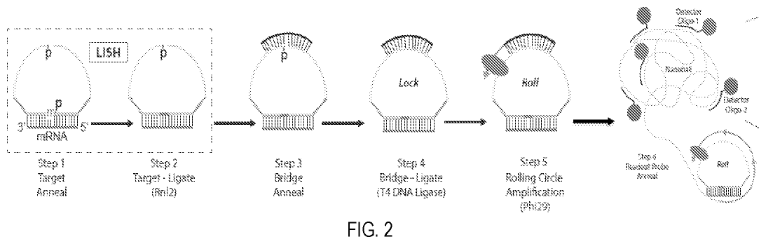

A schematic of LISH-Lock'n'Roll probe sets is shown in FIG. 1. The LISH-Lock'n'Roll probe set is composed of a 3′ acceptor probe and a 5′ donor probe (LnR probe set). The 3′ terminus of the acceptor probe set is composed of two ribonucleic acid bases at the 3′ end, which foster high efficiency ligation by the T4 RNA ligase, Rnl2. The 5′ donor probe is phosphorylated at the 5′ end. The probe set's targeting sequences are designed to anneal adjacent to one another on the RNA target. The targeting sequences can be roughly 20 nucleotides, but may also be substantially longer or shorter. Only when the LnR probes are annealed adjacent to one another on a target sequence can they be ligated together via Rnl2. This requirement that the ligation probes anneal to adjacent sequences provides a high level of assay specificity. Additionally, each acceptor and donor probe feature one or two 30-nucleotide detector sequences and a 17-nucleotide bridge sequence (FIG. 1). The LISH-Lock'n'Roll workflow is illustrated in FIG. 2. In step 1 and 2, the LnR acceptor and donor probes anneal to the target RNA sequence, followed by ligation with Rnl2. Excess probes are then washed away. After adjacent donor and acceptor probe have become ligated, will the two probe halves present the complete 34-nucleotide bridge sequence (17nt from each probe), which is subsequently hybridized by the bridge primer (step 3) and ligated by T4 DNA ligase (step 4). At this stage, the probe sets have been ligated at both ends, completing a circle, such that due to the twist of the double helix, locks it into place around the target mRNA. Phi29 polymerase is then added to the tissue, enabling rolling circle amplification (RCA) to take place in situ, as it is primed by the annealed bridge primer that was used for circularization (step 5). The RCA product is in essence a ‘nanoball’ of single stranded DNA containing many copies of the detector sequences. Due to the extensive crosslinking of the surrounding tissue, the nanoball remains trapped in a position that approximates the position of the templating RNA molecule. Following completion of RCA, fluorescently labeled oligos (detector probes) are annealed to the complementary detector sequences, of which there are now many spatially localized copies (step 6). The tissue is now ready to be processed for imaging.

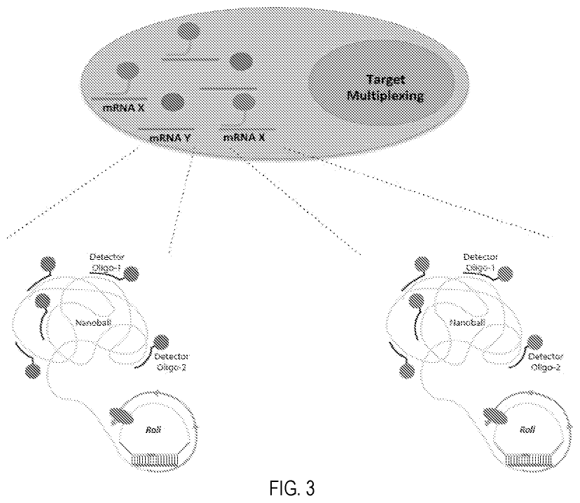

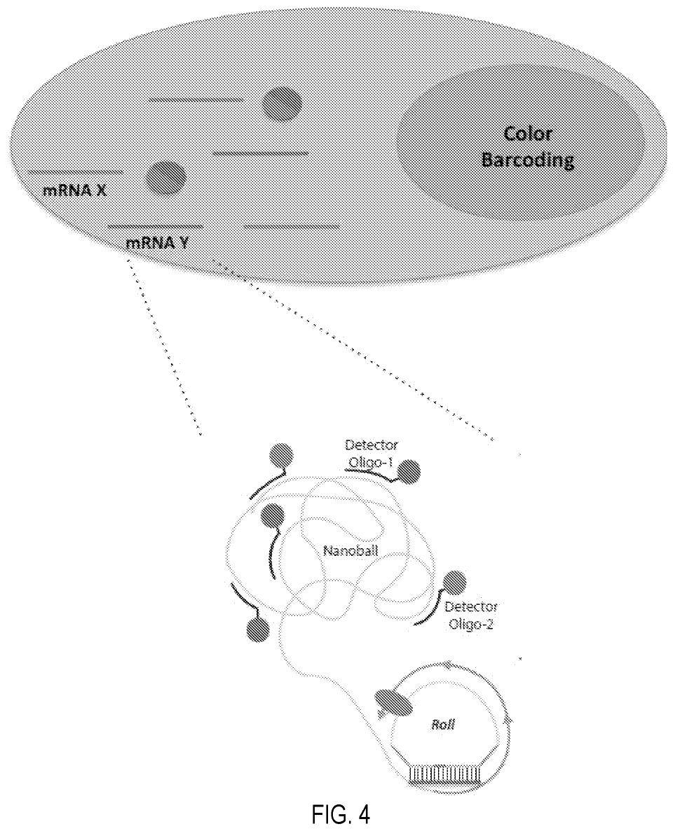

Probe sets are typically designed to have 1 to 4 unique detector sequences. FIG. 3 illustrates target multiplexing, which is the use of probe sets that target different mRNA transcripts using distinct detector sequences. FIG. 4 illustrates multiplexing based on color barcoding, wherein a probe set has two or more distinct detector sequences for simultaneous binding of two or more distinctly colored detector probes. Color barcoding enables a greater level of combinatorial multiplexing, as well as the opportunity for encoding error-correcting color combinations. Target multiplexing, combined with color barcoding, enables simultaneous spatial quantification of many different mRNA transcripts. As an example, a panel of LnR probe sets with two different detector sequences per probe set, and five uniquely colored detector probes, can be used to simultaneously measure more than 15 targets during a single cycle of imaging. Additional rounds of probe removal and detector probe hybridization can exponentially amplify the level of multiplexing achievable.

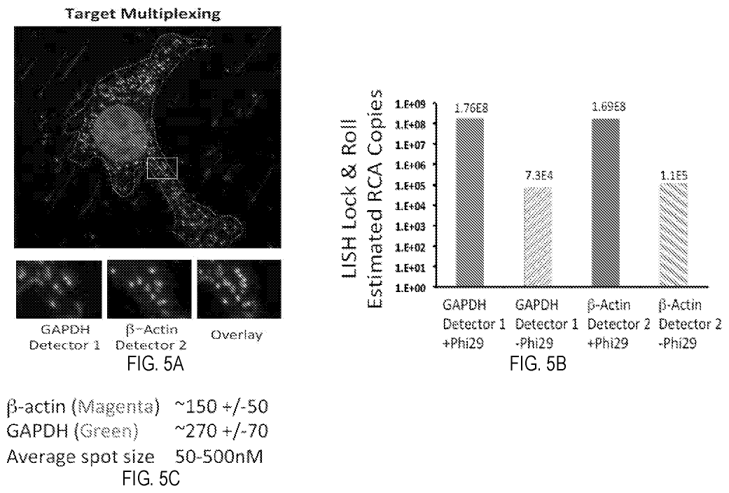

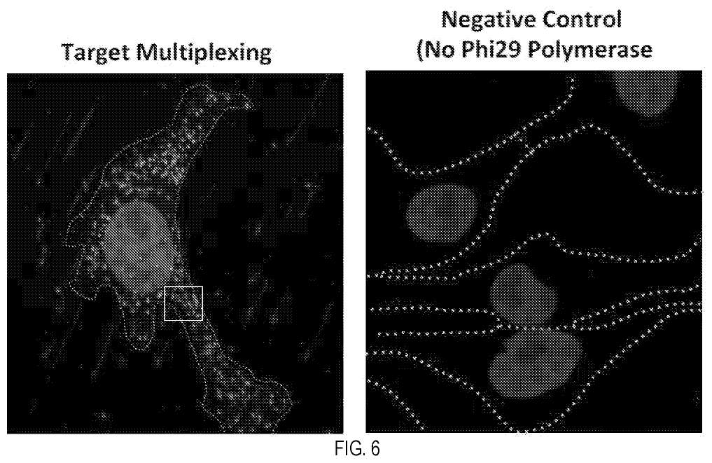

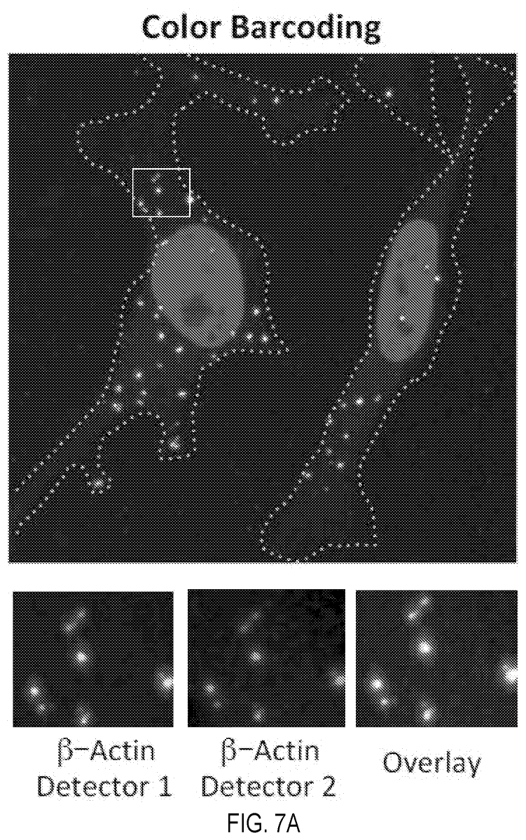

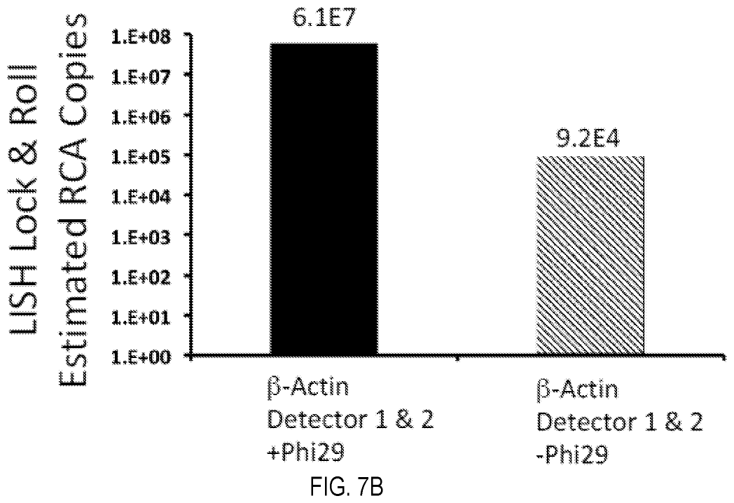

To determine the efficiency and specificity of LISH-Lock'n'Roll target multiplexing, the present inventors designed probe sets targeting GAPDH and β-actin for use in fixed HeLa cells as a model tissue (FIG. 5). Each probe set could be bound by only one of two spectrally distinct fluorophore-labeled detector probes, detector-1 (GAPDH, Alexa-488 labeled oligo) and detector-2 (β-actin, Alexa-647 labeled oligo) (FIG. 5A). The zoomed images (white box in main image) showing the individual detector probes when overlaid, revealed no spatial overlap of the individual detector probes. Phi29-dependent amplification of the locked circle was >1,000-fold higher than unamplified samples when the products were measured by qPCR using primers specific to the individual targeting sequences (FIG. 5B). Average spots per cell and average spot diameter were calculated using imageJ software (FIG. 5C). There were 150+/−50 spots/cell for β-actin and 270+/−70 spots/cell for GAPDH with both probe sets producing spot sizes of 50-500 nM in diameter. No spots were detectable when Phi29 was omitted (FIG. 6) revealing the high degree of specificity achievable with the LISH-Lock'n'Roll method. To determine the efficiency and specificity of LISH-Lock'n'Roll color multiplexing, the present inventors designed a single probe sets targeting β-actin that had two distinct detector sequences, which were also tested in fixed HeLa cells (FIG. 7A). The RCA product arising from this single probe set was equivalently bound by both of the spectrally distinct fluorophore-labeled detector probes, detector-1 (Alexa-488 labeled) and detector-2 (Alexa-647 labeled). As expected, the zoomed images (white box in main image) revealed complete overlap of the two detector probes. In this experiment, Phi29-dependent amplification of the locked circle was >600-fold higher than the unamplified samples when products were measured by qPCR using primers specific to the individual targeting sequences (FIG. 7B). As an example, a panel of LnR probe sets with four different detector sequences per probe set (two per probe), and five uniquely colored detector probes, can be used to simultaneously measure 30 targets during a single cycle of imaging. Additional rounds of probe removal and detector probe hybridization can exponentially amplify the level of multiplexing achievable.

### III. LISH Lock'n'Roll Embodiments

It is understood that embodiments reciting the detection of RNA is applicable to the detection of DNA as if those embodiments were written for detection of DNA. Thus, in one aspect, the present invention provides compositions and methods for detecting ribonucleic acids. In another aspect, the present invention can be used to detect deoxyribonucleic acids.

In one embodiment, a method for detecting an immobilized target nucleic acid comprises the steps of (a) contacting a biological sample comprising the target nucleic acid in a reaction mixture with at least one probe set comprising (i) a first multi-partite probe comprising a 5′ phosphorylated donor probe and a first bridge probe, wherein the 5′ phosphorylated donor probe specifically hybridizes to the target nucleic acid; and (ii) a second multi-partite probe comprising a 3′ acceptor probe and a second bridge probe, wherein the 3′ acceptor probe specifically hybridizes to the target nucleic acid adjacent to the 5′ donor probe and the second bridge probe is 5′ phosphorylated; (b) incubating the reaction mixture of step (a) under conditions that permit hybridization of the at least one probe set to the target nucleic acid present in the biological sample; (c) washing away unbound probe sets; (d) ligating the 5′ phosphorylated donor probe and the 3′ acceptor probe; (e) contacting the reaction mixture with at least one bridge primer that specifically hybridizes to the first bridge probe and the second bridge probe, wherein the first bridge probe and the second bridge probe anneal to the bridge primer adjacent to each other; (f) ligating the first bridge probe and the second bridge probe thereby forming a circularized probe that is hybridized to the target nucleic acid; (g) amplifying the circularized probe by rolling circle amplification; and (h) detecting the target nucleic acid.

In another embodiment, contacting step (e) is performed prior to ligating step (d). Thus, a method for detecting an immobilized target nucleic acid can comprise the steps of (a) contacting a biological sample comprising the target nucleic acid in a reaction mixture with at least one probe set comprising (i) a first multi-partite probe comprising a 5′ phosphorylated donor probe and a first bridge probe, wherein the 5′ phosphorylated donor probe specifically hybridizes to the target nucleic acid; and (ii) a second multi-partite probe comprising a 3′ acceptor probe and a second bridge probe, wherein the 3′ acceptor probe specifically hybridizes to the target nucleic acid adjacent to the 5′ donor probe and the second bridge probe is 5′ phosphorylated; (b) incubating the reaction mixture of step (a) under conditions that permit hybridization of the at least one probe set to the target nucleic acid present in the biological sample; (c) washing away unbound probe sets; (d) contacting the reaction mixture with at least one bridge primer that specifically hybridizes to the first bridge probe and the second bridge probe, wherein the first bridge probe and the second bridge probe anneal to the bridge primer adjacent to each other; (e) ligating the 5′ phosphorylated donor probe and the 3′ acceptor probe; (f) ligating the first bridge probe and the second bridge probe thereby forming a circularized probe that is hybridized to the target nucleic acid; (g) amplifying the circularized probe by rolling circle amplification; and (h) detecting the target nucleic acid. In a further embodiment, the ligating steps (steps (e) and (f) in the immediately previous embodiment) are performed simultaneously with the same or different ligases. Contacting step (d) can also be performed prior to washing step (c).

In an alternative embodiment, the contacting step of the bridge primer and the ligating step of the first bridge probe and the second bridge probe are performed prior to ligating step of the 5′ phosphorylated probe and the 3′ acceptor probe. Thus, the method can comprise (a) contacting a biological sample comprising the target nucleic acid in a reaction mixture with at least one probe set comprising (i) a first multi-partite probe comprising a 5′ phosphorylated donor probe and a first bridge probe, wherein the 5′ phosphorylated donor probe specifically hybridizes to the target nucleic acid; and (ii) a second multi-partite probe comprising a 3′ acceptor probe and a second bridge probe, wherein the 3′ acceptor probe specifically hybridizes to the target nucleic acid adjacent to the 5′ donor probe and the second bridge probe is 5′ phosphorylated; (b) incubating the reaction mixture of step (a) under conditions that permit hybridization of the at least one probe set to the target nucleic acid present in the biological sample; (c) washing away unbound probe sets; (d) contacting the reaction mixture with at least one bridge primer that specifically hybridizes to the first bridge probe and the second bridge probe, wherein the first bridge probe and the second bridge probe anneal to the bridge primer adjacent to each other; (e) ligating the first bridge probe and the second bridge probe thereby forming a circularized probe that is hybridized to the target nucleic acid; (f) ligating the 5′ phosphorylated donor probe and the 3′ acceptor probe; (g) amplifying the circularized probe by rolling circle amplification; and (h) detecting the target nucleic acid.

The present invention can be used to detect multiple target nucleic acids. Each probe set targets a specific and distinct nucleic acid (the 5′ phosphorylated donor probe and the 3′ acceptor probe specifically hybridize (adjacent to each other) a particular target nucleic acid. In such multiplex embodiments, the at least one probe set is configured for multiplex detection of 1-30,000 distinct target nucleic acids. In other embodiments, the at least one probe set comprises a range of combined 1-20,000, 10-10,000, 20-5000, or 50-1000 probe sets. In particular embodiments, the at least one probe set is configured to detect the presence or absence of SNPs, mutations, novel splicing isoforms, fusions and the like.

In other embodiments, more than one probe set can be designed to bind different locations/regions of the same nucleic acid. The number of rolling circle amplification (RCA) products formed per target nucleic acid can be decreased by adding unligatable probe sequences to the biological sample. During amplification, the size of the RCA products formed can be decreased by incorporating dideoxynucleotides, or other chain terminators.

In certain embodiments, the multipartite probes range in size from 30-1000 nucleotides. In other embodiments, multipartite probe of the present invention may range in size from about 30 to about 1000 nucleotides, from about 25 to about 9000 nucleotides, about 30 to about 8000 nucleotides, about 25 to about 5000 nucleotides, about 40 to about 2000 nucleotides, about 50 to about 1000, or about 30 to about 200 nucleotides.

The target RNA can be a viral RNA, a bacterial RNA, a fungal RNA, a nematode RNA, a human RNA, a non-human mammal RNA, a non-mammalian animal RNA or combinations thereof.

In particular embodiments, the present invention is used to detect an immobilized target RNA. In a specific embodiment, the RNA is immobilized as part of a fixed biological sample comprising the target RNA. The fixed biological sample can comprise fixed tissue, frozen-fixed tissue, formalin fixed paraffin embedded tissue, adherent fixed cells, suspension fixed cells or fixed cells.

In some methods of the present invention, the fixed biological sample comprises cells and the location of the rolling circle amplification products in the sample is used to infer the type or phenotype of a cell or cells. In some methods of the present invention, the fixed biological sample is tissue processed into sections having a thickness of 1-1000, 10-900, 20-800, 30-500, or 40-200 microns.

In particular embodiments, RCA products can be immobilized within the sample by crosslinking the RCA product to the biological sample. Crosslinking may occur by applying a reagent to the RCA products, wherein the reagent is paraformaldehyde, formaldehyde, formalin, glutaraldehyde, osmium tetroxide, potassium dichromate, chromic acid, and potassium permanganate, and Hepes-glutamic acid buffer-mediated organic solvent fixative, or a combination thereof.

Bridge primers used in the present invention may include a reactive moiety and RCA products may be immobilized within the sample by the reactive moiety on the bridging primer.

In alternative embodiments, the target RNA is immobilized by capture prior to washing step (c). A labeled target RNA capture probe can be used including, but not limited to, biotin, diogexin, acrydite, haloalkane, or click chemistry. A capture element can include avidin, streptavidin, neutravidin, anti-digoxin antibodies, click chemistry, halo protein, or a combination thereof. A solid support can be used to capture target RNA, and can include magnetic material, polystyrene, agarose, silica, lateral flow strip, microfluidic chambers, or a combination thereof can be used in the immobilization process.

In certain embodiments, the 3′ acceptor probe comprises at least one 3′ terminal ribonucleotide.

In particular embodiments, the ligating step is performed using a ligase selected from the group consisting of T4 RNA Ligase 2 (Rnl2), a Chlorella virus DNA ligase (PBCV-1 DNA Ligase), a T4 DNA Ligase, derivatives thereof, and combinations thereof.

In particular embodiments, detecting step (h) comprises sequencing or hybridization.

In certain embodiments, detecting step (h) comprises sequencing of rolling circle amplification products. All or a portion of the rolling circle amplification products can be sequenced. In a specific embodiment, the probe set comprises a barcode unique to the target RNA and wherein sequencing of the barcode detects the target RNA. In a more specific embodiment, the sequencing comprises sequencing by synthesis or sequencing by ligation. In an even more specific embodiment, the method is performed in situ on a fixed sample. In an alternative embodiment, the sequencing comprises sequencing by synthesis, and the synthesized sequence creates a unique color barcode that detects the target RNA.

In other embodiments, detecting step (h) comprises sequencing the ligated sequence formed by the donor probe and the acceptor probe.

In yet another embodiment, detecting step (h) comprises contacting the reaction mixture with a detectably labeled detector probe that specifically hybridizes the ligated sequence formed by the donor probe and the acceptor probe.

In other embodiments, the first multi-partite probe and the second multi-partite probe each further comprise at least one detection probe. In a specific embodiment, the first multi-partite probe and the second multi-partite probe each comprise two detection probes. In an even more specific embodiment, the first multi-partite probe and the second multi-partite probe each comprise a spacer sequence between the two detection probes.

In such embodiments, detecting step (h) comprises contacting the reaction mixture with at least one detector probe that specifically hybridizes to the at least one detection probe and imaging the at least one detectable labeled detector probe. In particular embodiments, the at least one detector probe is detectably labeled. In a specific embodiment, the method can further comprise the step of identifying the location of the target RNA in the sample. In another embodiment, the method further comprises the step of quantifying the target RNA in the sample.

Examples of detector probes used in the present invention include fluorescently labeled nucleic acid sequences in the range of 10 to 100 nucleotides bound to a detection element. The detector probes are fluorescently labeled with a fluorescent probe such as a fluorophore, fluorescent protein, quantum dot, biotin, digoxin, heavy metal mass tag, surface-enhanced Raman scattering tag, or peroxidase enzyme.

In further embodiments, multiple rounds of hybridization and stripping of detection probes can be performed. In a specific embodiment, the detection probes hybridize to detection sequences present in the probe set. In another specific embodiment, the detection probes hybridize to bar codes present in the probe set. In a non-limiting embodiment, RCA products are prepared on, for example, 1000 targets, creating nanoballs of DNA in the tissue. Ten rounds of hybridization with fluorescent probes are conducted. The probes are stripped off and the process is repeated until all of the targets are detected. If the probe set comprises multiple detection probes, then different combinations of fluorescent detector probes can be used to identify the different target nucleic acids.

In particular embodiments, amplification step (g) is performed using a strand displacing DNA polymerase. In specific embodiments, the strand displacing DNA polymerase comprises Phi29 polymerase or Bst polymerase.

### IV. LISH Lock'n'Roll Kits

Any of the compositions described herein may be comprised in a kit. In a non-limiting example one or more multi-partite probes of the present invention including 3′ acceptor and 5′ donor probes, one or more bridging primer, one or more DNA polymerase, one or more ligase, and one or more detector probes.

In one embodiment, a kit comprises a probe set comprising (i) a first multi-partite probe comprising a 5′ phosphorylated donor probe and a first bridge probe, wherein the 5′ phosphorylated donor probe specifically hybridizes to a target nucleic acid; and (ii) a second multi-partite probe comprising a 3′ acceptor probe and a second bridge probe, wherein the 3′ acceptor probe specifically hybridizes to the target nucleic acid adjacent to the 5′ donor probe and the second bridge probe is 5′ phosphorylated. It is understood that the kit can comprises multiple probe sets targeting distinct RNAs. In other embodiments, more than one probe set can bind to different locations/regions of the same target.

In another embodiment, the kit further comprises a bridge primer that specifically hybridizes to the first bridge probe and the second bridge probe, wherein the first bridge probe and the second bridge probes anneal to the bridge primer adjacent to each other.

In other embodiments, the first multi-partite probe and the second multi-partitie probe each further comprise at least one detection probe. In a specific embodiment, the first multi-partite probe and the second multi-partite probe each comprise two detection probes. In a more specific embodiment, the first multi-partite probe and the second multi-partite probe each comprise a spacer sequence between the two detection probes.

In other embodiments, the kit further comprises at least one detector probe that specifically hybridizes to the at least one detection probe. In specific embodiments, the at least one detector probe is detectably labeled.

In particular embodiments, the multipartite probes range in size from 30-1000 nucleotides. In other embodiments, the 3′ acceptor probe comprises at least one 3′ terminal ribonucleotide.

The kit can further comprise a ligase for ligating the 5′phosphorylated donor probe and the 3′ acceptor probe. More specifically, the ligase comprises T4 RNA Ligase 2 (Rnl2), a Chlorella virus DNA ligase (PBCV-1 DNA Ligase), a T4 DNA Ligase, and derivatives thereof.

The kit can further comprise a ligase for ligating the first bridge probe and the second bridge probe to form a circularized probe that is hybridized to the target nucleic acid.

In other embodiments, the kit further comprises a strand displacing DNA polymerase for amplifying by rolling circle amplification a circularized probe formed by ligating the first bridge probe and the second bridge probe and hybridized to the target nucleic acid. The strand displacing DNA polymerase can comprise Phi29 polymerase or Bst polymerase.

The kits may comprise a suitably sized aliquot of any of the compositions comprised herein and, in some cases, one or more additional agents such as buffers, for example. The component(s) of the kits maybe packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing any of the compositions described herein and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means within the kit.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

### LISH-Lock'n'Roll Probe Design

LISH-Lock'n'Roll probe pairs were designed to incorporate the following sequences. A first bridge probe comprises 5′-AGATCGGAAGAGCACAC-3′ (SEQ ID NO:1) and a second bridge probe comprises 5′-/5PHOS/-GGAGCTGTCGTTCACTC-3′ (SEQ ID NO:2), respectively. The first, second, third and fourth detector sequences were 5′-CAAGTATGCAGCGCGATTGACCGTCTCGTT-3′ (SEQ ID NO:3), 5′-CGCAACGCTTGGGACGGTTCCAATCGGATC-3′ (SEQ ID NO:4), 5′-ACAAATCCGACCAGATCGGACGATCATGGG-3′ (SEQ ID NO:5), and 5′-CAAGTATGCAGCGCGATTGACCGTCTCGTT-3′ (SEQ ID NO:6), respectively. The GAPDH target specific acceptor and donor sequences were 5′-TTGAGCACAGGGTACTTTrArT-3′ (SEQ ID NO:7) (‘r’ before nucleotides 19 and 20 indicate ribonucleotide) and 5′-/5Phos/-tgatggtacatgacaaggtg-3′ (SEQ ID NO:8) respectively, and the β-actin target specific acceptor and donor sequences were 5′-AAGGTGTGCACTTTTATTrCrA-3′ (SEQ ID NO:9) (‘r’ before nucleotides 19 and 20 indicate ribonucleotide) and 5′-/5Phos/-ACTGGTCTCAAGTCAGTGTA-3′ (SEQ ID NO:10). The bridge oligo, separate from the LISH-Lock'n'Roll probe pairs had the following sequence, 5-AGTGAACGACAGCTCCGTGTGCTCTTCCGA*T*C-3′ (SEQ ID

NO:11), with the asterisks indicating nucleotides that contained a phophorothioate bond substitution, which rendered them less susceptible to degradation by nucleases. Detector probe-1 complementary to detector sequence 1 was synthesized with a 5′-ATTO-647 fluorescent tag. Detector probe-4 complementary to detector sequence 4 was synthesized with a 5′-Alexa-488 fluorescent tag. In general, the target specific sequences can be designed using the guidelines published in reference.¹⁷⁻¹⁸ All probes and oligos were synthesized by Integrated DNA Technologies (Coralville, Iowa 52241, USA). Probe pairs were mixed in equimolar amounts to create simplified multiplex panels, which were aliquoted and stored at −80° C., and then diluted to a stock concentration of 1 uM per probe (10× final working concentration) for use in LISH-Lock'n'Roll assays.

### Example 2

### LISH-Lock'n'Roll

Samples consisted of adherent cells grown to ˜80% confluency on 18 mm #1 circular poly-lysine coated coverslips (ThermoFisher, Waltham, Mass. 02451, USA). Samples were fixed for 15 min with 1% paraformaldehyde (ThermoFisher) followed by three washes with 1X-PBS. Sample were permeabilized for 20 min in 1×-PBS, 0.1% Triton X-100. Next, samples were treated for 10 min with 0.1N HCl followed by three washes in 1×-PBS. Samples were incubated with 100 uL of LISH-Lock'n'Roll probe set panels diluted to 100 nM in hybridization buffer (6×-SSC, 10% formamide) and incubated for 2 hours at 45° C. Samples were washed three times in room temperature hybridization buffer followed by a single buffer exchange wash with T4 RNA Ligase reaction buffer (50 mM Tris-HCl, 10 mM MgCl₂, 5 mM DTT, 1 mM ATP, pH 7.6 @ 25° C.). Samples were incubated for 2 hours at 37° C. in 200 uL with T4 RNA Ligase enzyme (Qiagen, Hilden, Germany) diluted to 0.375 U/uL in T4 RNA Ligase reaction buffer. Samples were washed 3× in room temperature hybridization buffer followed by incubation at 37° C. for 1 hour with the bridge oligo diluted to 200 nM in 1×-PBS. Samples were washed 3× in room temperature 1× PBS followed by a single buffer exchange wash with room temperature T4 DNA ligase reaction buffer (50 mM Tris-HCl, 10 mM MgCl₂, 10 mM DTT, 1 mM ATP, pH 7.6 @ 25° C.). Samples were incubated for 1 hour at 37° C. in 200 uL with T4 DNA enzyme (New England Biolabs, Ipswich, Mass. 01938, USA) diluted to 50 U/uL in T4 DNA Ligase reaction buffer. Samples were washed in 1×-PBS followed by a single buffer exchange in 4° C. Phi29 reaction buffer (50 mM Tris-HCl, 10 mM (NH₄)₂SO₄, 4 mM Dithiothreitol, 10 mM MgCl₂, pH 7.5 @ 25° C.). Samples were then incubated for 2 hours at 30° C. in 200 uL with Phi29 Polymerase enzyme (Lucigen Corporation, Middleton, Wis. 53562, USA) diluted to 1 U/uL in Phi29 reaction buffer supplemented with dNTPs (final concentration 0.8 mM total nucleotides). Samples were washed 3× in 2×-SSC and incubated in the dark at room temperature for 30 min in 200 uL with detector probes diluted to 500 nM in 6×-SSC. Samples were washed 3× in 6×-SSC and fixed onto standard microscope slides with ProLong Gold antifade reagent containing DAPI (ThermoFisher). Finally, samples were imaged on a Zeiss LSM 880 with Airyscan (Oberkochen, Germany) with images post-processed using ImageJ (National Institutes of Health, USA).

### Example 3

### LISH-Lock'n'Roll Guides Cancer Treatment and Establishes a Prognosis

A patient with metastatic melanoma undergoes a surgical procedure to resect a tumor in order to investigate the composition of the immune microenvironment in proximity to the cancer cells. The resected tissue is preserved by fixation in formalin and embedding in paraffin wax; thin sections of the tissue block are cut and mounted on a microscope slide. A LISH-Lock'n'Roll panel has been designed to detect and distinguish about 100 different RNA molecules, many of which are known to be associated with response to immunotherapy, many of which are specific to certain cell populations, and some of which are housekeeping genes to be used for data normalization. After the LISH-Lock'n'Roll assay is performed and the data analyzed, it is determined that in this particular melanoma microenvironment, high levels of infiltrating CD8+ T cells are in the vicinity of tumor cells, and that the tumor cells themselves are expressing high levels of the immunosuppressive molecule Programmed death-ligand 1 (PD-L1). The patient is informed that they have a high likelihood of responding to a PD-L1 (or PD-1) checkpoint inhibitor therapy, such as atezolizumab, nivolumab, etc., and the patient is administered this regimen with a favorable outcome.

### Example 4

### LISH-Lock'n'Roll as a Diagnostic Test for Inclusion Body Myositis (IBM)

IBM is a chronic, debilitating, progressive inflammatory myopathy, which can be challenging to diagnose. The role of RNA misprocessing is increasingly recognized for its role in the pathology of the disease; the expectation in the field is that mRNA mis-splicing in particular will become increasingly utilized in disease diagnosis. Nuclear exclusion of the splicing factor TAR DNA-binding protein 43 (TDP43) and the expression of the cryptic exons it suppresses, are emerging as a hallmark of IBM. A patient suspected of having IBM would typically undergo a muscle biopsy as part of their diagnostic workup. Similar to Example 1, the tissue is preserved and sectioned for pathological examination. A TDP-43 cryptic exon detection panel comprising LISH-Lock'n'Roll probes are utilized to search for cells that appear to have lost the ability to splice out cryptic exons. It is expected that export of cryptic exon including mRNAs into the cytosol may precede extranuclear accumulation of TDP-43, and thus may serve as a more sensitive diagnostic test for IBM. If cells are identified that harbor LISH-Lock'n'Roll products corresponding to exported mRNAs containing cryptic exons (due to the color barcode design associated with such probes), then the diagnosis of IBM can be made with confidence.

### Example 5

### LISH-Lock'n'Roll Used to Perform Genetic Screens for Biomedical Research Applications

There are many ways in which LISH-Lock'n'Roll based analyses can be useful in the setting of biomedical research. One illustrative example is the use of combinatorial tracking of genetic constructs using ‘barcode’ sequences, which can be deciphered using LISH-Lock'n'Roll probe sets. Consider that cells have been transformed with a lentiviral library encoding a large set of CRISPR-Cas9 guide RNA (gRNA) sequences. Also expressed from the same vector is an RNA molecule that includes one or more sequences that alone or in combination are uniquely associated with each gRNA sequence. Live cell imaging is used to observe the migration patterns of each cell, at baseline and in the presence of different candidate chemotherapies at different doses. After the observation period is completed, the cells are fixed in place and the LISH-Lock'n'Roll assay is performed such that the pattern of spots is used to identify the LISH-Lock'n'Roll barcode and thus the gRNA expressed by each cell. This information is thus used to link the function of the gene that is targeted by the gRNA with its impact on the behavior of the cells at baseline and in the presence of the candidate chemotherapy. Such insight might be used in establishing combinatorial cancer therapies, for example.

### REFERENCES

1. Crosetto N, Bienko M & van Oudenaarden A (2015) Spatially resolved transcriptomics and beyond. Nat Rev Genet 16, 57— 66.

2. Lee J H (2017) Quantitative approaches for investigating the spatial context of gene expression. Wiley Interdiscip Rev Syst Biol Med 9, e1369.

3. Cheow L F, Courtois E T, Tan Y, Viswanathan R, Xing Q, Tan R Z, Tan D S, Robson P, Loh Y H, Quake S R et al. (2016) Single-cell multimodal profiling reveals cellular epigenetic heterogeneity. Nat Methods 13, 833— 836.

4. Rozenblatt-Rosen O, Stubbington M J T, Regev A & Teichmann S A (2017) The human cell atlas: from vision to reality. Nature 550, 451-453.

5. Saper, C. B. (2009). A guide to the perplexed on the specificity of antibodies. Journal of Histochemistry & Cytochemistry, 57(1), 1-5.

6. Belzil, V. V., Gendron, T. F., & Petrucelli, L. (2013). RNA-mediated toxicity in neurodegenerative disease. Molecular and Cellular Neuroscience, 56, 406-419.

7. O'Rourke, J. R., & Swanson, M. S. (2009). Mechanisms of RNA-mediated disease. Journal of Biological Chemistry, 284(12), 7419-7423.

8. Raj A, van den Bogaard P, Rifkin S A, van Oudenaarden A & Tyagi S (2008) Imaging individual mRNA molecules using multiple singly labeled probes. Nat Methods 5, 877-879.

9. Lubeck E, Coskun A F, Zhiyentayev T, Ahmad M & Cai L (2014) Single-cell in situ RNA profiling by sequential hybridization. Nat Methods 11, 360-361.

10. Shah S, Lubeck E, Zhou W & Cai L (2017) seqFISH accurately detects transcripts in single cells and reveals robust spatial organization in the hippocampus. Neuron 94, 752-758.e1.

11. Chen K H, Boettiger A N, Moffitt J R, Wang S & Zhuang X (2015) RNA imaging. Spatially resolved, highly multiplexed RNA profiling in single cells. Science 348, aaa6090.

12. Lee J H, Daugharthy E R, Scheiman J, Kalhor R, Yang J L, Ferrante T C, Terry R, Jeanty S S, Li C, Amamoto R et al. (2014) Highly multiplexed subcellular RNA sequencing in situ. Science 343, 1360— 1363.

13. Smith, A. M., Heisler, L. E., St. Onge, R. P., Farias-Hesson, E., Wallace, I. M., Bodeau, J., . . . & Nislow, C. (2010). Highly-multiplexed barcode sequencing: an efficient method for parallel analysis of pooled samples. Nucleic acids research, 38(13), e142-e142.

14. Klein A M, Mazutis L, Akartuna I, Tallapragada N, Veres A, Li V, Peshkin L, Weitz D A & Kirschner M W (2015) Droplet barcoding for single-cell transcriptomics applied to embryonic stem cells. Cell 161, 1187-1201.

15. Stahl P L, Salmen F, Vickovic S, Lundmark A, Navarro J F, Magnusson J, Giacomello S, Asp M, Westholm J O, Huss M et al. (2016) Visualization and analysis of gene expression in tissue sections by spatial transcriptomics. Science 353, 78-82.

16. Tang F, Barbacioru C, Wang Y, Nordman E, Lee C, Xu N, Wang X, Bodeau J, Tuch B B, Siddiqui A et al. (2009) mRNA-Seq whole-transcriptome analysis of a single cell. Nat Methods 6, 377-382.

17. Larman, H. B., Scott, E. R., Wogan, M., Oliveira, G., Torkamani, A., & Schultz, P. G. (2014). Sensitive, multiplex and direct quantification of RNA sequences using a modified RASL assay. Nucleic acids research, 42(14), 9146-9157.

18. Credle, J. J., Itoh, C. Y., Yuan, T., Sharma, R., Scott, E. R., Workman, R. E., & Miller, H. (2017). Multiplexed analysis of fixed tissue RNA using Ligation in situ Hybridization. Nucleic acids research, 45(14), e128-e128.

1. A method for detecting an immobilized target ribonucleic acid (RNA) comprising the steps of:
contacting a biological sample comprising the target RNA in a reaction mixture with at least one probe set comprising (i) a first multi-partite probe comprising a 5′ phosphorylated donor probe and a first bridge probe, wherein the 5′ phosphorylated donor probe specifically hybridizes to the target RNA; and (ii) a second multi-partite probe comprising a 3′ acceptor probe and a second bridge probe, wherein the 3′ acceptor probe specifically hybridizes to the target RNA adjacent to the 5′ donor probe and the second bridge probe is 5′ phosphorylated;
b. incubating the reaction mixture of step (a) under conditions that permit hybridization of the at least one probe set to the target RNA present in the biological sample;
c. washing away unbound probe sets;
d, ligating the 5′ phosphorylated donor probe and the 3′ acceptor probe;
e. contacting the reaction mixture with at least one bridge primer that specifically hybridizes to the first bridge probe and the second bridge probe, wherein the first bridge probe and the second bridge probe anneal to the bridge primer adjacent to each other;
f. ligating the first bridge probe and the second bridge probe thereby forming a circularized probe that is hybridized to the target RNA;
g. amplifying the circularized probe by rolling circle amplification; and
h. detecting the target RNA.

2. The method of claim 1, wherein the biological sample is fixed.

3. The method of claim 2, wherein the fixed biological sample comprises fixed tissue, frozen-fixed tissue, formalin fixed paraffin embedded tissue, adherent fixed cells, suspension fixed cells or fixed cells.

4. The method of claim 1, wherein the target RNA is immobilized by capture prior to step (c).

5. The method of claim 1, wherein the 3′ acceptor probe comprises at least one 3′ terminal ribonucleotide.

6. The method of claim 1, wherein step (e) is performed prior to step (d).

7. The method of claim 6, wherein ligating steps (d) and (f) are performed simultaneously.

8. The method of claim 1, wherein steps (e) and (f) are performed prior to step (d).

9. The method of claim 1, wherein ligating step (d) is performed using a ligase selected from the group consisting of T4 RNA Ligase 2 (Rnl2), a Chlorella virus DNA ligase (PBCV-1 DNA Ligase), a T4 DNA Ligase, derivatives thereof, and combinations thereof.

10. The method of claim 1, wherein detecting step (h) comprises sequencing of rolling circle amplification products.

11. The method of claim 10, wherein the probe set comprises a barcode unique to the target RNA and wherein sequencing of the barcode detects the target RNA.

12. The method of claim 11, wherein the sequencing comprises sequencing by synthesis or sequencing by ligation.

13. (canceled)

14. (canceled)

15. The method of claim 1, wherein detecting step (h) comprises sequencing the ligated sequence formed by the donor probe and the acceptor probe.

16. The method of claim 1, wherein detecting step (h) comprises contacting the reaction mixture with a detectably labeled detector probe that specifically hybridizes the ligated sequence formed by the donor probe and the acceptor probe.

17. The method of claim 1, wherein the first multi-partite probe and the second multi-partitie probe each further comprise at least one detection probe.

18. (canceled)

19. The method of claim 18, where the first multi-partite probe and the second multi-partite probe each comprise a spacer sequence between the two detection probes.

20. The method of claim 17, wherein detecting step (h) comprises contacting the reaction mixture with at least one detectably labeled detector probe that specifically hybridizes to the at least one detection probe and imaging the at least one detectable labeled detector probe.

21. The method of claim 20, further comprising the step of identifying the location of the target RNA in the sample.

22. The method of claim 20, further comprising the step of quantifying the target RNA in the sample.

23.-27. (canceled)

28. A kit comprising:
a probe set comprising (i) a first multi-partite probe comprising a 5′ phosphorylated donor probe and a first bridge probe, wherein the 5′ phosphorylated donor probe specifically hybridizes to a target RNA; and (ii) a second multi-partite probe comprising a 3′ acceptor probe and a second bridge probe, wherein the 3′ acceptor probe specifically hybridizes to the target RNA adjacent to the 5′ donor probe and the second bridge probe is 5′ phosphorylated.

29.-41. (canceled)
